(19) United States
(12) Patent Application Publication
(10) Pub. No.: US 2012/0097160 A1
(43) Pub. Date: Apr. 26, 2012

(54) AGGLOMERATE FORMULATIONS INCLUDING ACTIVE PHARMACEUTICAL AGENTS WITH TARGETED PARTICLE SIZES

(75) Inventors: Preetanshu Pandy, Springfield, NJ (US); Sai Prasanth Chamarthy, Springfield, NJ (US); Jennifer Lynn Wylie, Cranford, NJ (US)

(73) Assignee: SCHERING CORPORATION, Kenilworth, NJ (US)

(21) Appl. No.: 13/266,067

(22) PCT Filed: Apr. 23, 2010
(86) PCT No.: PCT/US2010/032228

§ 371 Date: Jan. 9, 2012

Related U.S. Application Data
(60) Provisional application No. 61/172,340, filed on Apr. 24, 2009.

(51) Int. Cl. A61K 31/56; A61P 11/00; A61M 15/00; A61K 9/16

(52) U.S. Cl. 128/203.15; 424/400; 514/169

(57) ABSTRACT

Various embodiments of the present invention provide for an agglomerate comprising at least one active pharmaceutical agent and at least one excipient; wherein at least about ninety percent of the at least one active pharmaceutical agent have a particle size of less than about 2 μm.

### FIELD OF THE INVENTION

Various embodiments of the present invention relate to dry powder inhalers and, more particularly, to agglomerates that yield a desirable fine particle fraction.

### BACKGROUND

Drug delivery to the lungs can be accomplished with dry powder inhalers (DPIs), metered dose inhalers, and nebulizers. The majority of DPIs are passive, meaning they are ‘breath-actuated’ devices where the patient provides the energy to aerosolize the powder during the inhalation. In order to deposit drug in the respiratory tract, DPIs deliver micron-sized drug particles having an aerodynamic diameter of approximately 1-5 μm. Particles of this size have a high surface area and a large number of contact points between particles. The dominant interparticle interactions for such systems are Van der Waals and Columbic interactions. DPI formulations have proved challenging since micronized powders tend to be cohesive and flow poorly, both of which result in poor aerosolization efficiency and delivery of the drug.

Common types of DPIs include an inhaler with a micronized powder in a packet or capsule, a carrier formulation based DPI or an agglomerate formulation based DPI. In the carrier-based system, micronized drug is mixed with a coarse excipient, typically between 60 and 90 microns. α-Lactose monohydrate is the most widely used carrier, although alternative carriers, such as sorbitol, xylitol and mannitol, have been studied. In a carrier-based system, the micronized drug adheres to the larger carrier particle. When the particles are entrained in the airstream during an inhalation, the drug separates from the surface of the carrier and is inhaled while the larger carrier particle impacts in the oropharynx and is cleared.

Another formulation approach is the agglomerate-based system. In this technique, micronized drug may be agglomerated with an excipient as used in PULMICORT TURBOHALER® dry powder inhaler (AstraZeneca, Wilmington, Del.) Alternatively, micronized drug may be combined with micronized excipient as used in ASMANEX TWISTHALER® dry powder inhaler (Schering-Plough, Kenilworth, N.J.) and are formulated into agglomerates as described in U.S. Pat. No. 6,503,537, which is incorporated herein in its entirety. During the patient's inhalation, turbulence and collisions between agglomerates and the inhaler walls break these agglomerates into fine drug and excipient particles.

A major difference between a carrier-based formulation and agglomerate-based formulation is that for the agglomerate-based formulation, the micronized drug as well as the micronized excipient gets inhaled into the deep lung, whereas, in carrier based systems, the large carrier particles do not reach the lung because they generally get stuck in the throat and other areas of the body before the lung. Thus, agglomerate-based systems have unique challenges since most of the powder from the agglomerate is inhaled into the lung. Generally, it is desirable to inhale the least amount of powder into the lung. Thus, it would be desirable to increase the efficiency of agglomerate based formulations by increasing the desirable fine particles (fine particle fraction or FPF) of the formulation that can reach the target areas of the lung to treat various respiratory diseases, such as asthma and COPD and to reduce the total amount of powder that needs to be inhaled from the DPI.

### SUMMARY

Agglomerate formulations and methods that are capable of controlling and increasing the fine particle fraction of agglomerate-based DPI systems were surprisingly discovered. Specifically, it was found that higher efficiency agglomerate formulations with a higher fine particle fraction for a delivered dose of an agglomerate-based DPI increases with decreasing APA particle size. More specifically, a higher fine particle fraction surprisingly was obtained with agglomerates prepared with a drug substance that contained a smaller particle size.

These results trend in the opposite direction to what has been reported in the literature for carrier-based DPI formulations (Taki M., Marriott, Zeng X., Martin G., An investigation into the influence of particle size, drug-drug and drug-excipient interactions on the aerodynamic deposition of drugs aerosolized from single and combination dry powder inhalers, Respiratory Drug Delivery, 2008, 589-592). For example, in carrier-based systems, it has been reported that smaller APA particles lead to a net increase in interaction forces between APA and carrier particles. It is believed that smaller APA particle sizes make it more difficult for smaller individual APA particles to detach from the carrier particles during drug delivery, resulting in inhaled APA particles that selectively have larger particle size or are APA clumps of smaller particles and thus, a lower fine particle fraction. Thus, a priori, one skilled in the art would not try to decrease the particle size of drug substance used to prepare agglomerate based formulations since one skilled in the art would believe that such particle would produce larger particles/clumps upon actuation of a DPI and, undesirably, have a lower FPF. The present invention surprisingly found that APA with a smaller particles used in an agglomerate actually produce particles with a higher FPF when emitted upon actuation of a DPI.

Various embodiments of the present invention provide for an agglomerate comprising at least one active pharmaceutical agent and at least one excipient; wherein at least about ninety percent of the at least one active pharmaceutical agent have a particle size of less than about 2 μm. Additionally, the agglomerate may have at least about 50% of the at least one active pharmaceutical agent has a particle size of less than about 1 μm. A preferable excipient is a binder and may be lactose anhydrous NF. The agglomerate may have a hardness of at least about 9 mN, at least about 10 mN, at least about 13 mN or at least about 15 mN. The active pharmaceutical agent emitted dose from a dry powder inhaler may have a fine particle fraction of greater than about 30%, about 50%, about 60%, about 70%, about 75% or about 80%. Useful at least one active pharmaceutical agent include but are not limited to an anticholinergic, a corticosteroid, a long acting beta agonist, short acting beta agonist, a phosphodiesterase 4 inhibitor and combinations of two or more thereof.

Additional embodiments of the present invention provide for an agglomerate comprising at least one active pharmaceutical agent and lactose; wherein the at least about ninety percent of the at least one active pharmaceutical agent has a particle size of less than about 2 μm. Still other embodiments of the present invention include an agglomerate comprising at least one active pharmaceutical agent and at least one excipient; wherein one of the at least one active pharmaceutical agents has at least about ninety percent of its particles have a particle size less than about 2 μm and wherein a second active pharmaceutical agent has about ninety percent of its particles have a particle size is not less than about 2 μm. Further embodiments of the present invention include an agglomerate comprising at least one active pharmaceutical agent and lactose; wherein at least about ninety percent of the at least one active pharmaceutical agent has a particle size of less than about 2 μm and wherein the agglomerate has a hardness of at least 9 mN.

Still further embodiments of the present invention include a drug product comprising a dry powder inhaler device and at least one agglomerate comprising at least one active pharmaceutical agent and at least one excipient; wherein at least about ninety percent of the at least one active pharmaceutical agent has a particle size of less than about 2 μm. The one of the at least one active pharmaceutical agents has a Dv90 of less than 2 μm and a second of the at least one active pharmaceutical agent has a Dv90 of greater than 2 μm. The hardness of the agglomerate is at least about 9 mN, at least about 10 mN, at least about 13 mN or at least about 15 mN.

### BRIEF DESCRIPTION OF THE DRAWINGS

FIG. 1. Optical microscopy pictures of three agglomerate batches with varying APA particle size; (A) batch 1 (APA Dᵥ₅₀=0.92 μm), (B) batch 2 (APA Dᵥ₅₀=1.19 μm), and (C) batch 3 (APA Dᵥ₅₀=2.30 μm).

FIG. 2. Dispersed agglomerate microscopy pictures of three agglomerate batches with varying APA particle size; (A) batch 1 (APA Dᵥ₅₀=0.92 μm), (B) batch 2 (APA Dᵥ₅₀=1.19 μm), and (C) batch 3 (APA Dᵥ₅₀=2.30 μm).

FIG. 3. SEM images of agglomerates of BATCH 1 batch (APA Dᵥ₅₀=0.92 μm)

FIG. 4. SEM images of agglomerates of BATCH 2 batch (APA Dᵥ₅₀=1.19 μm)

FIG. 5. SEM images of agglomerates of BATCH 3 batch (APA Dᵥ₅₀=2.30 μm)

FIG. 6. Emitted dose (n=10) as a function of APA particle size

FIG. 7 Fine particle fraction obtained from ACI as a function of API particle size as measured by Sympatec.

FIG. 8. SEM of a typical agglomerate-based formulation

### DETAILED DESCRIPTION

The present invention surprisingly discovered agglomerate formulations and methods that are capable of controlling and increasing the fine particle fraction of agglomerate-based DPI systems. The present invention surprisingly discovered that the fine particle fraction of a delivered dose of an agglomerate-based DPI increases with decreasing APA particle size. A higher fine particle fraction was obtained with agglomerates prepared with a drug substance that was smaller in size. These APA particle size results trend in the opposite direction to what has been reported in the literature for carrier-based DPI formulations (Taki M., Marriott, Zeng X., Martin G., An investigation into the influence of particle size, drug-drug and drug-excipient interactions on the aerodynamic deposition of drugs aerosolized from single and combination dry powder inhalers, Respiratory Drug Delivery, 2008, 589-592). For carrier-based systems, it has been reported that smaller APA particles lead to a net increase in interaction forces between APA and carrier particles. It is believed that the smaller particle size APA particles make it more difficult for APA particles to detach from the carrier particles during drug delivery, resulting in inhaled particles that clump together and thus, has a bigger particle size and a lower fine particle fraction. Thus, a priori, one skilled in the art would not try to decrease the particle size of drug substance used to prepare agglomerate based particles since one skilled in the art would believe that such particle would produce larger particles upon actuation of a DPI and, undesirably, have a larger FPF. The present invention surprisingly found that APA with a smaller particles used in an agglomerate actually produce particles with a higher FPF when actuated from a DPI.

It is believed that this phenomenon could be due to several contributing factors (for example, particle shape, surface energy of particles, hardness and porosity of agglomerates). In various embodiments of the present invention, indentation data showed that agglomerates formed with an APA with a small particle size produced stronger agglomerates. It was expected that harder agglomerates would result in a lower fine particle fraction, however, it was surprisingly discovered that harder agglomerates, which had APAs with a smaller particle size, produced a higher fine particle fraction.

Dv stands for volume diameter. DvX is the volume diameter below which X percent of the log normal cumulative size distribution falls. Dv90 is the volume diameter below which 90 percent of the log normal cumulative size distribution falls. Dv50 is the volume diameter below which 50 percent of the log normal cumulative size distribution falls. Dv10 is the volume diameter below which 10 percent of the log normal cumulative size distribution falls. Thus, Dv90 is defined to mean that at least about ninety percent of the at least one active pharmaceutical agent have a particle size of less than a certain particle size. Thus, Dv50 is defined to mean that at least about fifty percent of the at least one active pharmaceutical agent have a particle size of less than a certain particle size.

Various embodiments of the present invention provide for an agglomerate useful in DPIs, wherein the agglomerate includes at least one excipient and at least one active pharmaceutical agent that have a Dv90 less than about 5 microns (μm), less than about 4 microns, less than about 3 microns (μm), less than about 2.5 microns (μm), less than about 2 microns (μm), less than about 1.8 microns (μm), less than about 1.7 microns (μm), less than about 1.5 microns (μm), less than about 1.3 microns (μm) or less than about 1 microns (μm).

Various embodiments of the present invention provide for an agglomerate useful in DPIs, wherein the agglomerate includes at least one excipient and at least one active pharmaceutical agent that has Dv50 of less than about 2 microns (μm), 1.8 microns (μm), less than about 1.7 microns (μm), less than about 1.5 microns (μm), less than about 1.3 microns (μm), less than about 1.2 microns (μm), less than about 1.1 microns (μm), less than about 1.0 microns (μm) or less than about 0.75 microns (μm).

Another requirement for an agglomerate particle-based DPI is that the agglomerate formulation must be hard enough not to prematurely separate prior to actuation of the DPI. The agglomerate formulation must be hard enough to withstand forces during product shipping and handling while it is idling in the reservoir in the DPI as well as throughout the manufacturing process. A priori, one skilled in the art would be led to believe agglomerates prepared with smaller particles would be stronger due to stronger forces associated with small particles and would therefore be harder to break into fine particles. This would in turn be expected to decrease the fine particle fraction of the formulation. This has been established in the literature for carrier-based systems (Taki M., Marriott, Zeng X., Martin G., An investigation into the influence of particle size, drug-drug and drug-excipient interactions on the aerodynamic deposition of drugs aerosolized from single and combination dry powder inhalers, Respiratory Drug Delivery, 2008, 589-592). Surprisingly, agglomerate formulations as claimed in various embodiments of the present invention were shown to have a higher fine particle fraction when formulated using a smaller API particle size.

Various embodiments of the present invention include at least one APA. Some embodiments may have two or three APAs. By varying the particle size of the various APAs used to make the agglomerate, the resulting emitted FPF dose from a DPI may be tailored to accommodate particular needs. For instance, it may be desirable to have an agglomerate formulation with one APA have a FPF of 30 or 40% whereas it may be desirable to have a second APA in the same agglomerate formulation with a FPF of 60 or 70%. By varying the particle size of the starting APA, this type of agglomerate formulation is now possible. A third and fourth APA with varying particle sizes may be also included in one agglomerate. Additionally, the particle size of the excipients included in a single agglomerate may be varied to tailor a desirable resulting emitted FPF from a DPI.

Such agglomerate formulations are useful in dry powder inhaler systems, such as the TWISTHALER®, sold by Schering-Plough.

Useful excipients include lactose, such as lactose anhydrous NF, lactose monohydrate or combinations thereof.

Several other embodiments provide for a dosing system comprising a DPI device and an agglomerate; wherein when the DPI device is actuated and the agglomerate is delivered, an actuated dose comprises a fine particle fraction of at least 30%, at least 40%, at least 50%, at least 60% at least 70%, at least 75%, or at least 80%.

An agglomerate in accordance with the present invention is a bound mass of small particulates. Agglomerates may include at least one first material and at least one excipient, such as a solid binder. The first material, in accordance with the present invention can be anything as the present invention can be used broadly to make free-flowing agglomerates for any application including, medicine, cosmetics, food and flavoring, and the like. Desirably, the first material is an active pharmaceutical agent or drug which is to be administered to a patient in need of some course of treatment.

Agglomerates of drug alone or with another substance may be utilized, such as those agglomerates described in U.S. Pat. No. 6,503,537, which is incorporated herein. Any method of agglomerating the solid binder and the pharmacologically active agent may be used. Useful agglomerating methods include those which can be accomplished without converting the amorphous content of the solid binder to a crystalline form, prematurely, and which does not require the use of additional binder, can be practiced in accordance with the present invention.

An agglomerate in accordance with the present invention is a bound mass of small particulates. The agglomerates include at least one first material and at least one solid binder. The first material, in accordance with the present invention can be anything as, indeed, the present invention can be used broadly to make free-flowing agglomerates for any application including, medicine, cosmetics, food and flavoring, and the like. However, preferably, the first material is an active pharmaceutical agent or drug which is to be administered to a patient in need of some course of treatment.

The active pharmaceutical agent may be administered prophylactically as a preventative or during the course of a medical condition as a treatment or cure. The active pharmaceutical agent or drug may be a material capable of being administered in a dry powder form to the respiratory system, including the lungs. For example, a drug in accordance with the present invention could be administered so that it is absorbed into the blood stream through the lungs. More preferably, however, the active pharmaceutical agent is a powdered drug which is effective to treat some condition of the lungs or respiratory system directly and/or topically.

Useful agglomerates include agglomerates ranging in size from between about 100 to about 1500 μm. The agglomerates may have an average size of between about 300 and about 1,000 μm. Useful agglomerates may have a bulk density which ranges from between about 0.2 to about 0.4 g/cm³ or between about 0.29 to about 0.38 g/cm³.

It is useful to have a tight particle size distribution. In this context, particle size refers to the size of the agglomerates. Preferably, no more than about 10% of the agglomerates are 50% smaller or 50% larger than the mean or target agglomerate size. For example, for an agglomerate of 300 μm, no more than about 10% of the agglomerates will be smaller than about 150 μm or larger than about 450 μm.

A useful method of preparing the agglomerates is described in U.S. Pat. No. 6,503,537, which is incorporated herein. Suitable methods involve mixing preselected amounts of one or more pharmacologically active agent(s) and the micronized, amorphous content containing, dry solid binder in a ratio of between about 100:1 and about 1:500; between about 100:1 and about 1:300 (drug:binder); between about 20:1 to about 1:20 or a ratio of about 1:3 to about 1:10 relative to the amount of the solid binder.

Useful agglomerates may have a strength which ranges from between about 50 mg and about 5,000 mg and most preferably between about 200 mg and about 1,500 mg. The crush strength was tested on a Seiko TMA/SS 120C Thermomechanical Analyzer available from Seiko Instruments, Inc. Tokyo, Japan, using procedures available from the manufacturer. It should be noted that strength measured in this manner is influenced by the quality and extent of the interparticulate crystalline bonding described herein. However, the size of the agglomerates also plays a role in the measured crush strength. Generally, larger agglomerates require more force to crush than do the smaller particles.

Various pharmaceutical active agents may be utilized. Suitable at least one active pharmaceutical agents include but are not limited to an anticholinergic, a corticosteroid, a long acting beta agonist, short acting beta agonist, a phosphodiesterase IV inhibitor. Suitable medicaments may be useful for the prevention or treatment of a respiratory, inflammatory or obstructive airway disease. Examples of such diseases include asthma or chronic obstructive pulmonary disease.

Suitable anticholinergics include (R)-3-[2-hydroxy-2,2-(dithien-2-yl)acetoxy]-1-1[2-(phenyl)ethyl]-1-azoniabicyclo[2.2.2]octane, glycopyrrolate, ipratropium bromide, oxitropium bromide, atropine methyl nitrate, atropine sulfate, ipratropium, belladonna extract, scopolamine, scopolamine methobromide, methscopolamine, homatropine methobromide, hyoscyamine, isopriopramide, orphenadrine, benzalkonium chloride, tiotropium bromide, GSK202405, an individual isomer of any of the above or a pharmaceutically acceptable salt or hydrate of any of the above, or a combination of two or more of the above.

Suitable corticosteroids includes mometasone furoate; beclomethasone dipropionate; budesonide; fluticasone; dexamethasone; flunisolide; triamcinolone; (22R)-6.alpha., 9.alpha.-difluoro-11.beta., 21-dihydroxy-16.alpha., 17.alpha.-propylmethylenedioxy-4-pregnen-3,20-dione, tipredane, GSK685698, GSK799943 or a pharmaceutically acceptable salt or hydrate of any of the above, or a combination of two or more of the above.

Suitable long acting beta agonist include carmoterol, indacaterol, TA-2005, salmeterol, formoterol, or a pharmaceutically acceptable salt or hydrate of any of the above, or a combination of two or more of the above. Suitable short acting beta agonist include albuterol, terbutaline sulfate, bitolterol mesylate, levalbuterol, metaproterenol sulfate, pirbuterol acetate or a pharmaceutically acceptable salt or hydrate of any of the above, or a combination of two or more of the above.

Suitable phosphodiesterase IV inhibitors include cilomilast, roflumilast, tetomilast, 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-L-proline, ethyl ester or a pharmaceutically acceptable salt or hydrate of any of the above, or a combination of two or more of the above.

In certain embodiments of the present invention the at least one active pharmaceutical agent includes a corticosteroid, such as mometasone furoate. Mometasone furoate is an anti-inflammatory corticosteroid having the chemical name, 9,21-Dichloro-11(beta), 17-dihydroxy-16(alpha)-methylpregna-1,4-diene-3,20-dione 17-(2 furoate). It is practically insoluble in water; slightly soluble in methanol, ethanol, and isopropanol; soluble in acetone and chloroform; and freely soluble in tetrahydrofuran. Its partition coefficient between octanol and water is greater than 5000. Mometasone can exist in various hydrated, crystalline and enantiomeric forms, e.g., as a monohydrate.

Several of these compounds could be administered in the form of pharmacologically acceptable esters, salts, solvates, such as hydrates, or solvates of such esters or salts, if any. The term is also meant to cover both racemic mixtures as well as one or more optical isomers. The drug in accordance with the present invention can also be an inhalable protein or a peptide such as insulin, interferons, calcitonins, parathyroid hormones, granulocyte colony-stimulating factor and the like. “Drug” as used herein may refer to a single pharmacologically active entity, or to combinations of any two or more, an example of a useful combination being a dosage form including both a corticosteroid and a β-agonist. A preferred active pharmaceutical agent for use in accordance with the present invention is mometasone furoate.

To be topically effective in the lungs or the upper and/or lower airway passages, it is desirable that the active pharmaceutical agent be delivered as particles of about 10 μm or less. See Task Group on Lung Dynamics, Deposition and Retention Models For Internal Dosimetry of the Human Respiratory Tract, Health Phys., 12, 173, 1966. The ability of a dosage form to actually administer free particles of these therapeutically effectively sized particles is the fine particle fraction. Fine particle fraction is, therefore, a measure of the percentage of bound drug particles released as free particles of drug having a particle size below some threshold during administration. Fine particle fraction can be measured using a multi-stage liquid impinger manufactured by Copley Instruments (Nottingham) LTD using the manufacturer's protocols. In accordance with the present invention, an acceptable fine particle fraction is at least 10% by weight of the drug being made available as free particles having an aerodynamic particle size of 6.8 μm, or less, measured at a flow rate of 60 liters per minute.

The amount of drug administered will vary with a number of factors including, without limitation, the age, sex, weight, condition of the patient, the drug, the course of treatment, the number of doses per day and the like. For mometasone furoate, the amount of drug delivered per dose, i.e. per inhalation, will generally range from about 10.0 μg to about 10,000 μg. Doses of 25 μg, 50 μg, 75 μg, 100 μg, 125 μg, 150 μg, 175 μg, 200 μg, 250 μg, 300 μg, 400 μg and/or 500 μg are preferred.

The solid binder in accordance with the present invention can be any substance which can be provided in, or reduced to, a particle size which is roughly congruent with the size of the particles of the active pharmaceutical agent as previously described. For example, agglomerates of mometasone furoate anhydrous USP will preferably be provided having particles of at least 80%≦5 μm and at least 95%≦10 μm (measured by volume distribution). The solid binder, such as anhydrous lactose, NF will be provided having particles of at least 60%≦5 μm, at least 90% under 10 μm, and at least 95%≦20 μm. The average particle size is roughly the same for both and is less than 10 μm.

Suitable solid binders include polyhydroxy aldehydes, polyhydroxy ketones, and amino acids. Preferred polyhydroxy aldehydes and polyhydroxy ketones are hydrated and anhydrous saccharides including, without limitation, lactose, glucose, fructose, galactose, trehalose, sucrose, maltose, raffinose, mannitol, melezitose, starch, xylitol, mannitol, myoinositol, their derivatives, and the like.

Particularly useful amino acids include glycine, alanine, betaine and lysine.

Percentages are expressed on a weight basis, unless the context clearly indicates otherwise. The mention of any specific drug substance in this specification or in the claims is intended to encompass not only the base drug, but also pharmaceutically acceptable salts, esters, hydrates and other forms of the drug. Where a particular salt or other form of a drug is mentioned, it is contemplated that other salts or forms can be substituted.

### EXAMPLES

### Materials and Methods

The APA used in this study belongs to a class of PDE-4 (Phosphodiesterase 4) inhibitors. Anhydrous lactose was used as the excipient in the formulation. The lactose was micronized using a jet-mill to an average particle size close to 2 μm.

Micronization of APA

The APA first underwent a delumping process by being passed through a Quadro mill (Quadro Comil Co., model 197AS). A portion of the quadro-milled material was used for batch manufacturing (referred as batch 3) while the remaining material (batch 1 and 2) was subsequently micronized using a jet mill micronizer (Jet Pulverizer Co., micron master 4 inch) at different feed rates and pressures as outlined in Table 1 to produce different APA particle sizes. After micronization, the powder particle size distributions were determined using a Sympatec particle size analyzer (Sympatec GmbH) with a HELOS™ and RODOS™ attachment. Particle size results (Dᵥ₁₀, Dᵥ₅₀, Dᵥ₉₀) for each of the three APA lots can be found in Table 1.

TABLE 1
Micronization conditions of APA lots and corresponding
particle size data

| | Pressure | Feed rate | Dᵥ₁₀ | Dᵥ₅₀ | Dᵥ₉₀ |
|---|---|---|---|---|---|
| Batch | (psi) | (g/min) | μm | | |
| 1 | 75 | 25 | 0.40 | 0.92 | 1.71 |
| 2 | 40 | 50 | 0.51 | 1.19 | 2.46 |
| 3 | N/A | N/A | 0.85 | 2.30 | 7.30 |

Batch Manufacturing

Three batches were manufactured at a batch size of 400 g, each containing one of the three lots of micronized APA (Batch 1, 2 and 3) and micronized lactose anhydrous. The APA concentration in these batches was 14.7% w/w. The micronized lactose and APA are blended together in a 2 qt. V-blender shell equipped with an intensifier bar to impart high-shear to the micronized powders (not free flowing due to adhesion, cohesion, and electrostatic effects). After blending, the powder is formulated into free-flowing agglomerates using a sieve-shaker to produce agglomerates with an average diameter of 500 μm and a bulk density of approximately 0.35 g/ml (3). Processing parameters such as blending time, screen size of sieve shaker, agglomeration time, curing time and conditions are controlled to produce agglomerates with desired physical properties. These agglomerates are filled into Schering-Plough's TWISTHALER® device.

Bulk Physical Characteristics of Agglomerates

The physical characteristics of bulk agglomerates were evaluated by optical microscopy, SEM, indentation, and particle size distribution. Optical micrographs of both the intact agglomerates and agglomerates dispersed within an oily non-solvent were taken to observe the surface morphologies and APA particle shape. Intact or undispersed agglomerates were observed under a stereomicroscope with oblique illumination. The photographs were captured using a digital camera through a range of magnifications (40-100×). Dispersed agglomerates were observed under polarized light microscope at a magnification of 100×.

Agglomerates were tested using indentation techniques (CSM Instruments, Needham, Mass.) to quantify their hardness. A 2 mm radius flat-tipped punch probe was used to indent the agglomerates. The loading and unloading rates used were 25.0 mN/min. Agglomerates were placed on a flat surface and crushed slowly by the flat probe until the first ‘fail’ point (first point of force deflection observed in the indentation curves) was observed. This was used as an indicator of the hardness of agglomerates.

The particle size distributions of bulk agglomerates were determined using a Sympatec laser diffraction particle size analyzer equipped with a GRADIS (gravimetric dispersion) dry powder disperser and a vibratory feeder.

Mass Median Aerodynamic Diameter and Emitted Dose Uniformity

Andersen cascade analysis on the inhalers was conducted for the Batch 1, 2 and 3. A total of 5 individual inhalers were tested. A modified Andersen cascade impactor (ACI) apparatus consisting of a glass throat, a centering DPI inhaler adapter, a sample solvent filled (10 ml) pre-separator, seven impactor stages (−1 through 5), and a filter were assembled and tested to ensure an inspiratory flow rate of 60 liters per minute under continuous flow. The cut-off diameters of the seven plates listed in the order above were 8.6, 6.5, 4.4, 3.3, 2.0, 1.1, and 0.54 μm, respectively. The particles below 0.54 μm were collected on the filter.

The inhaler was actuated in the ACI for 2 sec. The mass of APA deposited on each stage was determined by HPLC. HPLC was operated under isocratic conditions with a mobile phase consisting of 40% acetonitrile and 60% water containing 0.5% trifluoroacetic acid at a flow rate of 1 ml/min. The column was temperature controlled at 40° C. and detection was by UV at 254 nm by an external standard method of measurement. The fine particle fraction for this study is defined as the percentage of particles under the particle size of 6.5 μm.

A total of 10 individual inhalers were tested for emitted dose. The dose emitted from each inhaler was collected in an apparatus consisting of a modified separatory funnel with a fitted-glass frit and a glass fiber filter. Single inhalation of the dose was collected per test run. Dose was drawn at an airflow rate of 60 l/min applied for 2 sec through a vacuum line in series with a flow control as per USP procedure recommendation. The collected dose was assayed by HPLC.

Results

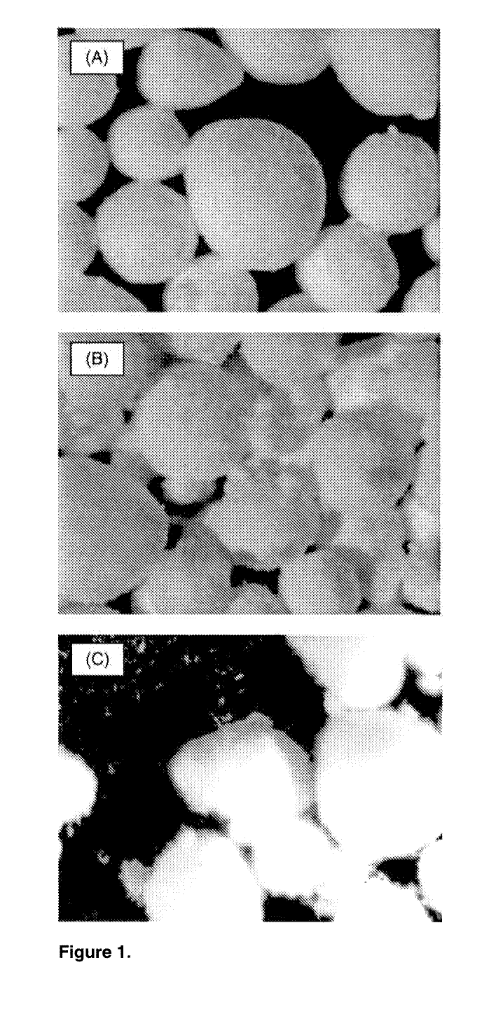

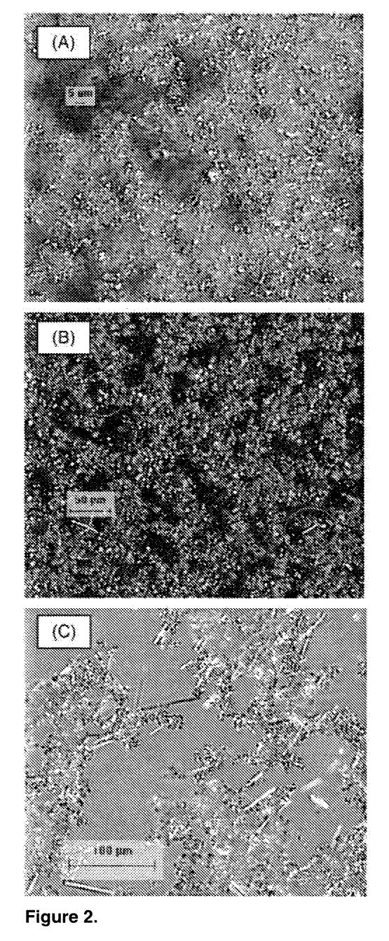

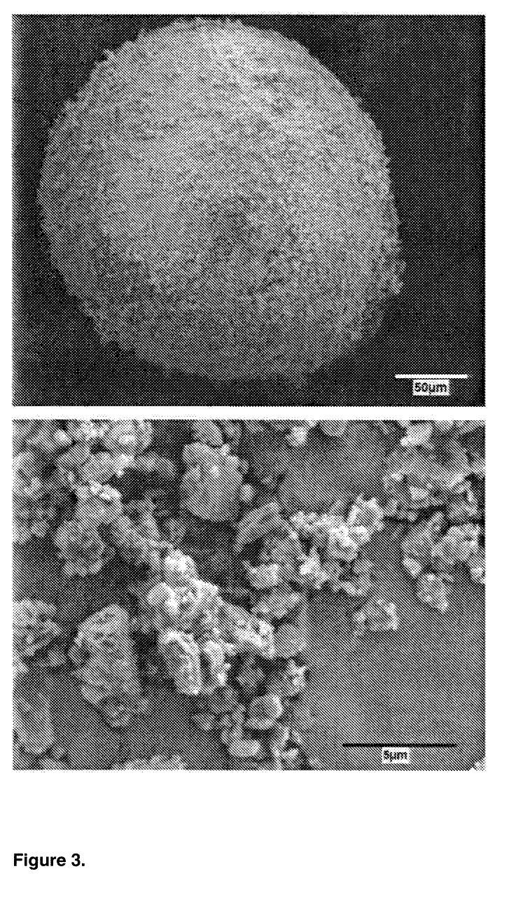

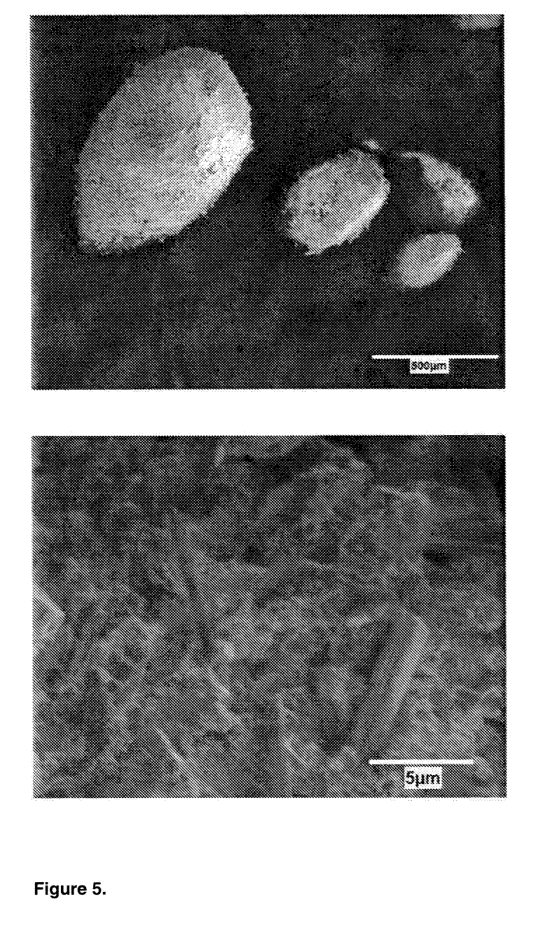

Optical micrographs of both the intact agglomerates and agglomerates dispersed within an oily non-solvent were taken. These micrographs reveal that intact agglomerates manufactured, the APA batch 2 have a smooth surface and appear spherical in shape (FIG. 1A). Agglomerates manufactured from APA batch 2 are less spherical in shape and show regions where rod-like APA particles protrude from the agglomerate surface (FIG. 1B). Lastly, agglomerates manufactured from Batch 3 APA batch show agglomerates containing many rod-like structures protruding from the agglomerates (FIG. 1C), and seem broken-up compared to other batches. Polarized light micrographs of the dispersed agglomerates confirmed the presence of completely micronized APA in batch 1 (FIG. 2A), while batch 2 revealed a few examples of rod-like APA around 10-50 μm in length (FIG. 2B). Batch 3 displayed numerous rod-like structures of APA, however, these rods were around 20-100 μm in length (FIG. 2C). SEM pictures of the agglomerates at different magnifications are presented in FIGS. 3-5 for the three batches. These pictures further confirm that the batch 1 is a well dispersed system, batch 2 has presence of some needle-shaped APA particles in it, whereas batch 3 has several needle-shaped APA particles in it. The microscopy results show that the harshest micronization condition (batch 1) APA produces agglomerates that are more spherical in shape and appear to be more uniform in size. The rod-like structures organized in a random orientation could result in increased fragility of the agglomerates by limiting the number of contact points where lactose and APA could adhere possibly leading to a decrease in adhesional forces.

TABLE 2
Agglomerate particle size as a function of APA particle size

| | Particle size, μm | | |
|---|---|---|---|
| APA Batch | Dᵥ₁₀ | Dᵥ₅₀ | Dᵥ₉₀ |
| 1 | 274.7 | 459.0 | 642.6 |
| 2 | 217.7 | 400.9 | 570.6 |
| 3 | 291.6 | 470.0 | 655.5 |

Table 2 shows the bulk agglomerate particle size distribution data obtained from Sympatec for the three batches. There seems to be no correlation between the average particle size of the bulk agglomerates and the initial APA particle size. On actuation of TWISTHALER® device the agglomerates, entrained in the airflow, follow a tortuous path in the device that helps them to break into the desired particle size range as they exit the inhaler. This means that the agglomerates have a certain inherent ‘hardness’ to be able to sustain the stress experienced during shipping and handling, and yet be able to break up to the desired particle size levels during inspiration. Controlling the physical characteristics (e.g. hardness) of such agglomerates can be critical to such a product. Indentation technique was evaluated to quantify the ‘hardness’ of these agglomerates. The first point of deflection observed in the indentation curves was marked as the force at which agglomerate first crushes. This was referred to as the ‘first fail’ value. The repeatability of the measurement checked for one sample (batch 3) by performing the test three times. The first fail values obtained were 8.71, 8.40 and 8.62 mN. The first fail values for batch 1 and batch 2 batches were estimated to be 13.90 and 9.67 mN respectively. Therefore, the indentation test showed that the agglomerates from batch 1 were the hardest, followed by batch 2 and then batch 3. These results confirm further the findings from optical microscopy and SEM images.

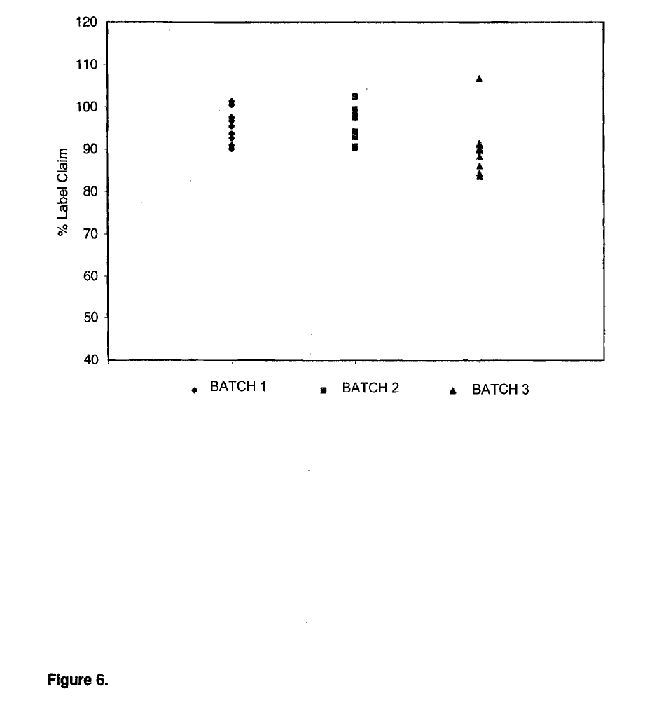

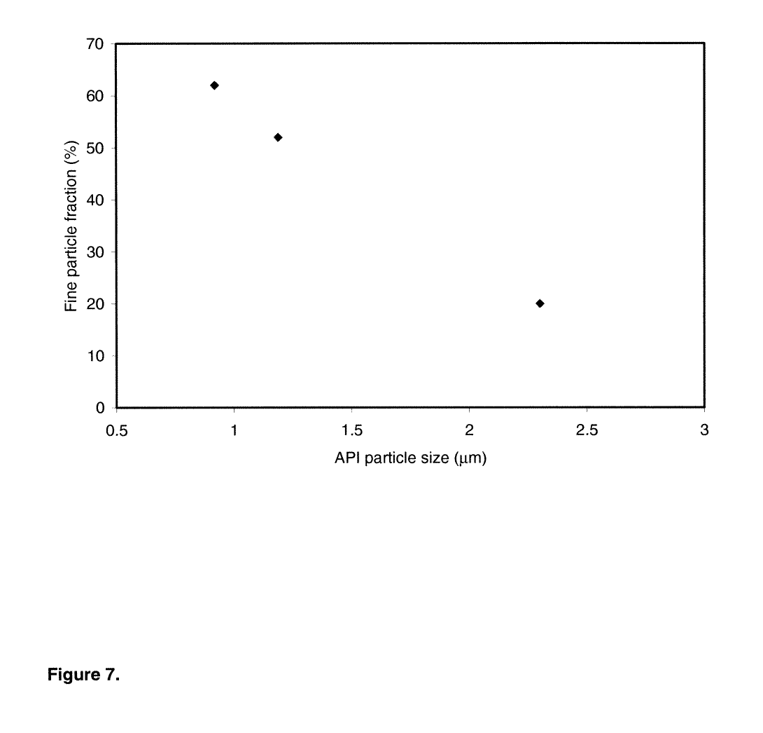

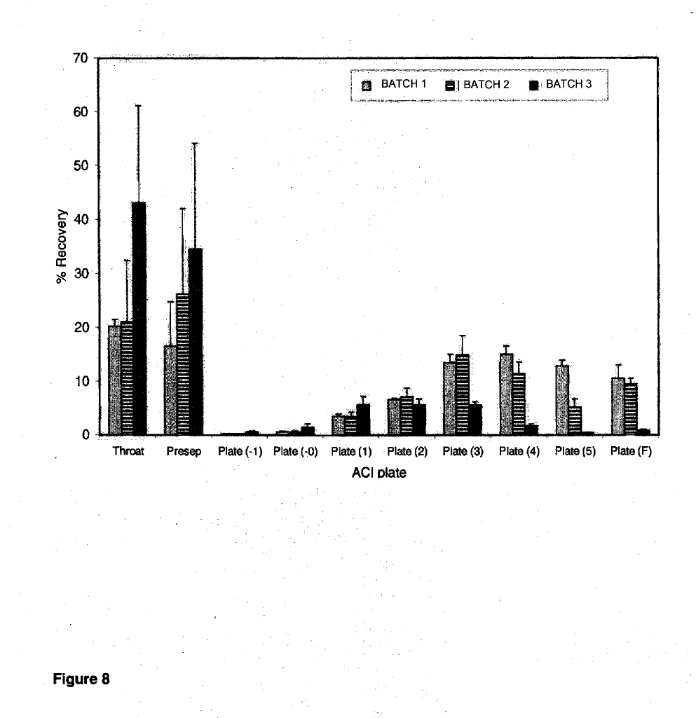

The performance of the formulation in the device was evaluated using emitted dose and aerodynamic particle size distribution. FIG. 6 shows the individual inhaler results obtained from emitted dose testing. The results indicate that all three sample lots meet the FDA guidance for emitted dose uniformity (EDU). All samples had individual emitted dose values within 80-120% of the label claim while having a mean between 85-115% (n=10). There was no observable trend with changing APA particle size, although the EDU values were observed to be generally lower and more variable for the batch 3. FIG. 7 shows the dependence of fine particle fraction (<6.5 μm in this study) as obtained by ACI as a function of APA particle size as measured by Sympatec. FPF is defined as the percent of the fine particle dose recovered in the fine particle range. Batch 1 and batch 2 both had high FPFs of 62% and 52%, respectively, while batch 3 had a lower FPF at 20%. FIG. 8 shows the aerodynamic particle size distribution of the drug particles exiting the TWISTHALER® for each of the three APA agglomerate batches. The ACI data are summarized in Table 3 with the MMAD (mass median aerodynamic diameter), GSD (geometric standard deviation). MMAD is defined as the median diameter of the particles based on mass and accounts for physical properties such as density and particle shape which can affect its aerodynamic flight characteristics. The aerodynamic diameter of a particle is equivalent to a unit density spherical particle having the same terminal settling velocity as the actual particle. MMAD is used to predict the settling site within the lungs and can be obtained using ACI testing. It was observed that as the APA particle size increases, the MMAD of the corresponding particles exiting the Twisthaler also increases. It should be noted that this does not mean that MMAD is solely a function of the APA particle size.

TABLE 3
ACI results summary as a function of APA particle size

| | APA particle | | |
|---|---|---|---|
| Batch | size, μm | MMAD, μm | GSD, μm |
| 1 | 0.92 | 1.35 | 2.18 |
| 2 | 1.19 | 1.49 | 2.21 |
| 3 | 2.30 | 3.17 | 2.21 |

It is interesting to note that a higher fine particle fraction is observed for the agglomerate batch (batch 1) that showed highest ‘hardness’ values, which may seem counter-intuitive at first. This means that fine particle fraction is not entirely a function of the hardness of agglomerates but there are other factors that influence it more strongly. First, it must be pointed out that the agglomerate hardness (as referred to in this study) is a macroscopic property (of the scale of agglomerate particle size, 200-800 μm), whereas fine particle fraction relates to the adhesion between drug and excipient and cohesion between drug particles at the APA and excipient particle size scale (1-2 μm in this case). An inverse relationship was very surprising.

As discussed in the introduction section, the tensile strength (σ) of agglomerate can be given by:

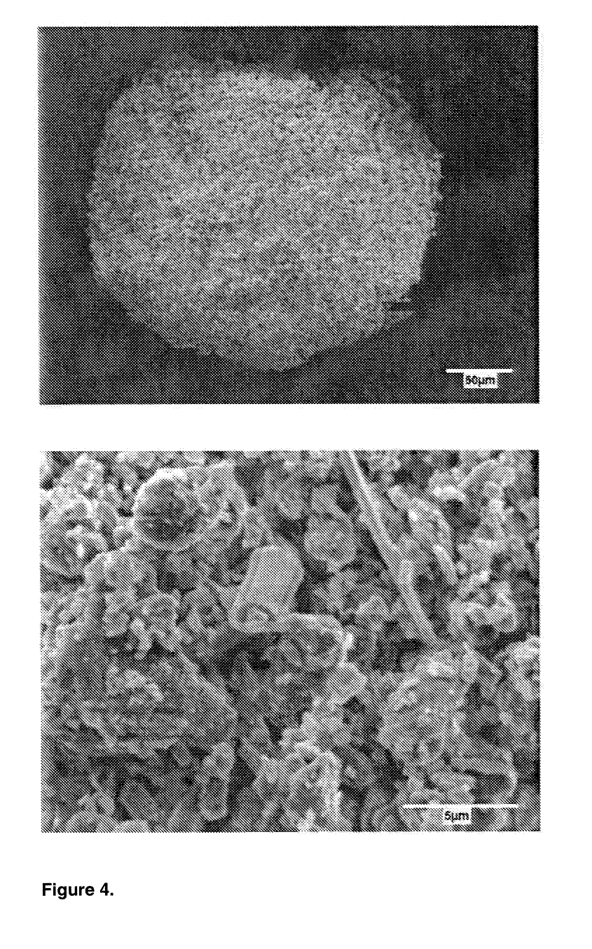

σ=15.60φ⁴R/D

where φ is the packing fraction, R is the work of fracture, and D is the particle diameter. This equation suggests that the strength of an agglomerate will increase with a reduction in APA particle size and an increase in packing fraction (stronger function). This is in agreement with the results from indentation testing, where agglomerates are found to be harder (higher tensile strength) for the case with smaller APA particle size. In addition, FIG. 3 shows less porous packing for the smaller APA particle size when compared to FIG. 4, which then reflects in the smaller APA particle size producing strongest agglomerates. Thus, the hardness results are in agreement with the equation presented above. However, this does not correlate directly to fine particle fraction obtained from ACI.

### CONCLUSIONS

This study was designed to determine the effects of APA particle size on the performance of an agglomerate-based dry powder inhaler system. APA particles were micronized at three different milling conditions and formulations were prepared from those micronized APA and micronized lactose in the form of agglomerates.

The results show that the fine particle fraction as obtained from ACI decreases with increasing APA particle size as measured by Sympatec. Indentation data show that the strongest agglomerates are formed with the smallest APA particle size. It was seen that fine particle fraction is not directly related to the agglomerate hardness.

The foregoing descriptions of various embodiments of the invention are representative of various aspects of the invention, and are not intended to be exhaustive or limiting to the precise forms disclosed. Many modifications and variations may occur to those having skill in the art. It is intended that the scope of the invention shall be fully defined solely by the appended claims.

1. An agglomerate comprising at least one active pharmaceutical agent and at least one excipient; wherein at least about ninety percent of the at least one active pharmaceutical agent have a particle size of less than about 2 μm.

2. The agglomerate of claim 1, wherein at least about 50% of the at least one active pharmaceutical agent has a particle size of less than about 1 μm.

3. The agglomerate of claim 1, wherein the at least one excipient is a binder.

4. The agglomerate of claim 1, wherein the at least one excipient is lactose anhydrous NF.

5. The agglomerate of claim 1, wherein the hardness of the agglomerate is at least 9 mN.

6. The agglomerate of claim 1, wherein the hardness of the agglomerate is at least 13 mN.

7. The agglomerate of claim 1, wherein the active pharmaceutical agent emitted dose from a dry powder inhaler has a fine particle fraction of greater than about 50%.

8. The agglomerate of claim 1, wherein at least one active pharmaceutical agent emitted dose from a dry powder inhaler has a fine particle fraction of greater than about 70%.

9. The agglomerate of claim 1, wherein the at least one active pharmaceutical agent is selected from the group consisting of an anticholinergic, a corticosteroid, a long acting beta agonist, short acting beta agonist, a phosphodiesterase 4 inhibitor and combinations of two or more thereof.

10. An agglomerate comprising at least one active pharmaceutical agent and lactose; wherein the at least about ninety percent of the at least one active pharmaceutical agent has a particle size of less than about 2 μm.

11. An agglomerate comprising at least one active pharmaceutical agent and at least one excipient; wherein one of the at least one active pharmaceutical agents has at least about ninety percent of its particles have a particle size less than about 2 μm and wherein a second active pharmaceutical agent has about ninety percent of its particles have a particle size is not less than about 2 μm.

12. The agglomerate of claim 10 wherein the agglomerate has a hardness of at least 9 mN.

13. A drug product comprising a dry powder inhaler device and at least one agglomerate according to claim 1.

14. The drug product of claim 13, wherein one of the at least one active pharmaceutical agents has a Dv90 of less than 2 μm and a second of the at least one active pharmaceutical agent has a Dv90 of greater than 2 μm.

15. The drug product of claim 13, wherein the hardness of the drug product is at least 9 mN.

16. The drug product of claim 13, wherein the hardness of the drug product is at least 13 mN.
